(19) United States
(12) Patent Application Publication
(10) Pub. No.: US 2020/0008737 A1
(43) Pub. Date: Jan. 9, 2020

(54) Transrectal Diagnostic Device

(71) Applicant: The Trustees of Columbia University in the City of New York, New York, NY (US)

(72) Inventors: Jong Hwan LEE, Seoul (KR); Hyun Keol KIM, Cresskill, NJ (US); Emerson LIM, New York, NY (US); Andreas H. HIELSCHER, Brooklyn, NY (US)

(73) Assignee: The Trustees of Columbia University in the City of New York, New York, NY (US)

(21) Appl. No.: 16/303,706

(22) PCT Filed: May 26, 2017
(86) PCT No.: PCT/US2017/034800

§ 371 Date: Nov. 21, 2018

Related U.S. Application Data
(60) Provisional application No. 62/344,584, filed on Jun. 2, 2016.
(60) Provisional application No. 62/342,670, filed on May 27, 2016.

(51) Int. Cl. A61B 5/00; A61B 8/12; A61B 8/13; A61B 8/00

(52) U.S. Cl. CPC A61B 5/4381; A61B 5/004; A61B 5/0073; A61B 5/0075; A61B 5/0084; A61B 5/0035; A61B 8/0833; A61B 8/13; A61B 8/4416; A61B 8/4461; A61B 8/4494; A61B 5/0037; A61B 8/12

(57) ABSTRACT

A probe and connected system provide ultrasound imaging and optical tomographic imaging of the prostate. A transrectal component carries ultrasound and optical sources and detectors movably connected to a reference element called a probe carrier that provide a reference frame to coregister anatomical and optical tomographic image data.

### BACKGROUND

Prostate cancer (PC) is the most common cause of cancer and the second most common cause of cancer deaths in American men. It is estimated that that there were 220,800 new cases of PC diagnosed and 27,540 deaths caused by PC in 2015. While the lifetime risk of PC for men in the United States is as high as 17%, only 3% of men will die of it. Moreover, one autopsy study found that 64% of men in their seventh decade of life had undiagnosed invasive PC but died of other causes.

While treatments for PC exist and can be effective at halting the progression of the disease, they also carry significant risks of undesirable side effects and complications. Such treatments include radical prostatectomy and radiation therapy. However, they can cause long-term incontinence, erectile dysfunction, proctitis, and cystitis. It is thus very desirable to not only detect PC early, but also to distinguish between cases of PC that require treatment and those that do not. Current risk assessment tools, however, suffer from various problems.

The diagnosis of diseases such as prostate cancer can be based on the standard histologic examinations together with a combination of prostate specific antigen (PSA) testing and digital rectal examination (DRE). However, these diagnosis methods have not been able to provide sufficient accuracy for detecting and localizing prostate cancer. The histologic examinations using image guided biopsy are subject to sampling errors and can also lead to adverse side effects such as bleeding, pain, infectious complications and so on. Though PSA testing shows high sensitivity, its low specificity requires the biopsy confirmation. DRE can access only restricted area near inner rectal wall and more than half of a prostate cannot be examined. Furthermore, non-invasive imaging modalities such as trans-rectal ultrasound (TRUS), CT and MRI show low sensitivity and specificity for staging prostate cancer.

### SUMMARY

Diffuse optical tomography (DOT) techniques can characterize the malignancy of tissue in the prostate based on the optical properties of examined tissue and chromophore concentrations. An optical probe that can perform DOT according to an exemplary embodiment can be used as an endo-rectal probe and allows for an increased number of measurement data points and easy co-registration of sources and detectors positions and TRUS images. The endorectal optical probe enables the rotation of the combined DOT and TRUS probes to acquire multi-angle data for the prostate cancer detection. The optical probe combined with a TRUS probe can be rotated based on a fixed position probe holder. At different angular steps, the DOT optical data and TRUS data can be obtained simultaneously. This rotational design can increase the measurement points and can generate a stack of TRUS images, which can be co-registered with optical measurement data. The increased data points and a priori structural information from TRUS data can improve the quality of the DOT imaging reconstruction results. More accurate assessment of prostate cancer using DOT imaging may be able to help in guiding early management decisions for patients.

Embodiments of the present disclosure provide a diagnostic device and methods that overcome the disadvantages of prior approaches for diagnosing PC. In an embodiment, the diagnostic device is an endo-rectal probe that provides simultaneous diffuse optical tomography (DOT) imaging and trans-rectal ultrasound (TRUS) at multiple angles, providing multiple views of the prostate in the diagnostic process. The device includes a TRUS probe and an DOT probe that forms a sleeve around the TRUS probe, with the sleeve and the TRUS probe locked together into one monolithic object. The device also includes a reference frame, and the monolithic object (TRUS and DOT sensing) is rotated within the reference frame to provide multiple views at known angles. The reference frame provides a frame of reference for angular rotation of the monolithic object when the device is inserted into the rectum of the patient.

Embodiments of the present disclosure provide rotatable probe that provides the combined ability to perform ultrasound examination at the same time as diffuse optical tomography with a single medical device.

A probe and connected system provide ultrasound imaging and optical tomographic imaging of the prostate. A transrectal component carries ultrasound and optical sources and detectors movably connected to a reference element called a probe carrier that provide a reference frame to coregister anatomical and optical tomographic image data.

Objects and advantages of embodiments of the disclosed subject matter will become apparent from the following description when considered in conjunction with the accompanying drawings. The foregoing summary does not comprehend all the embodiments or inventive aspects of the disclosed subject matter and serves merely to assist the reader.

### BRIEF DESCRIPTION OF THE DRAWINGS

The accompanying drawings, which are incorporated herein and constitute part of this specification, illustrate exemplary embodiments of the disclosure, and, together with the general description given above and the detailed description given below, serve to explain the features of embodiments of the disclosed subject matter. The accompanying drawings have not necessarily been drawn to scale. Where applicable, some features may not be illustrated to assist in the description of underlying features.

FIG. 1 illustrates a perspective view of a transrectal diagnostic device according to an exemplary embodiment of the disclosure.

FIG. 2 illustrates components of the transrectal diagnostic device according to an embodiment of the disclosure.

FIG. 3A is a blown-up view of an elongate sensor carrier according to an exemplary embodiment of the disclosure.

FIG. 3B illustrates a transrectal diagnostic device according to another exemplary embodiment.

FIGS. 4A and 4B illustrate a probe carrier according to an exemplary embodiment of the disclosure.

FIG. 5 illustrates another exemplary embodiment of a transrectal diagnostic device.

FIG. 6 illustrates a probe carrier according an exemplary embodiment of the disclosure.

FIG. 7A illustrates a side view of an exemplary embodiment of the transrectal diagnostic device.

FIG. 7B illustrates a close-up view of a region of FIG. 7A.

FIG. 8 illustrates a perspective view of a transrectal diagnostic device according to an exemplary embodiment of the disclosure.

FIG. 9 illustrates a cross-sectional view of the transrectal diagnostic device of FIG. 8.

FIG. 10 illustrates an exemplary embodiment of an ultrasound transducer assembly.

FIG. 11 illustrates a schematic of a cross-sectional view of a gear in an exemplary embodiment of a bimodal probe.

FIG. 12 illustrates a diagnostic system according to an exemplary embodiment of the disclosure.

FIG. 13 illustrates a device according to an exemplary embodiment of the disclosure.

### DETAILED DESCRIPTION

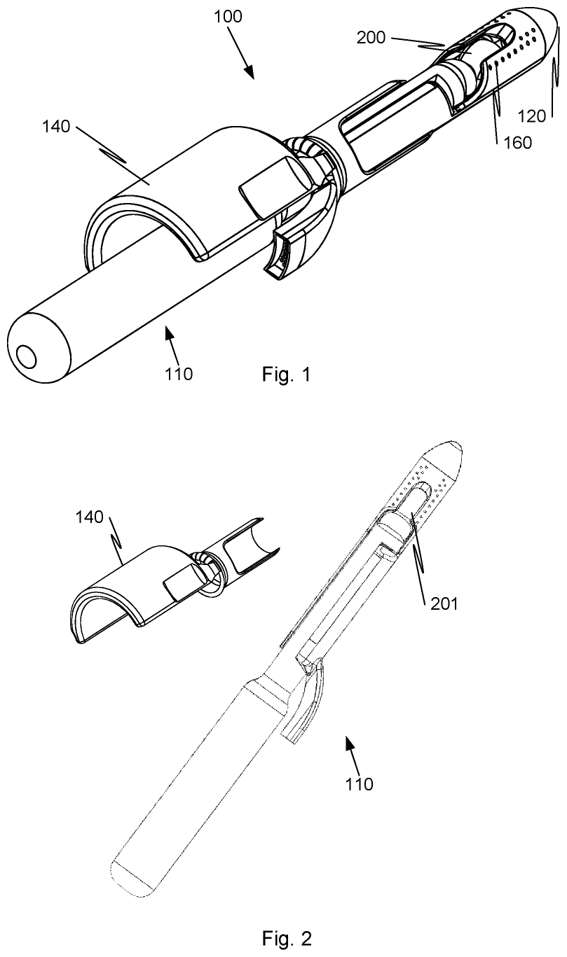

Referring to FIG. 1, an exemplary embodiment of transrectal diagnostic device 100 is shown. The transrectal diagnostic device 100 includes an elongate sensor carrier 110 and a probe carrier 140. The elongate sensor carrier 110 is inserted axially into a cavity formed in the central region of the probe carrier 140, as can be seen in FIG. 2, which illustrates the elongate sensor carrier 110 separated from the probe carrier 140.

In an exemplary embodiment, the elongate sensor carrier 110 carries two different types of sensors—acoustic and optical. Referring again to FIG. 1, the acoustical type of sensor may include one or more ultrasound transducers, in the present example, a single ultrasound transducer 200 which is positioned near the distal end 120 of the elongate sensor carrier 110. The optical type of sensor may include an optical tomography type transducer including an array of sources and detectors defined by the distal ends (i.e., the terminal ends) 160 of an array of optical fibers (only one terminal end of a fiber is indicated at each of multiple locations). The optical fibers themselves are not visible in the present drawings but may be understood to be routed within the body of the elongate sensor carrier 110. The optical fibers pass through the elongate sensor carrier 110 where the proximal ends terminate at their proximal ends at an optical fiber connector 310. The distal ends 160 of the optical fibers 150 may terminate at multiple apertures on a distal end 120 of the elongate sensor carrier 110 on a side of the distal end 120 as shown. The distal end 120 may be generally tapered to ease insertion into a body cavity. Diffusers may be provided at the fiber distal ends 160 or other light conditioners. At any given time, the fiber distal ends 160 function as sources and detectors by having light conveyed to, and received by, each of the fiber distal ends. Thus, in discussion of processing, the fibers may be called sources and detectors.

Although not illustrated, it will be understood that the source optical fibers that extend from distal ends 160 to the optical fiber connector 310 may be replaced with discrete light emitting sources, such as light emitting diodes, at the location of the apertures 160. Similarly, the detectors may be replaced with optical sensors which output an electrical signal representative of the light received. Thus, the optical fibers in the optical probe body may be replaced with electrical wires passing to connector 310.

As shown in FIG. 2, the ultrasound transducer is exposed to the inspected tissue through an ultrasound transducer window 201 positioned over the transducer 200. In use, it may be desirable to have the transducer 200 in contact with the patient's tissue or acoustically connected through a matching gel. An acoustic matching gel may be applied in the space around and on top of the transducer window 201 so that it fills any space between the transducer 200 and the patient's tissue to avoid or reduce an impedance mismatch and improve the quality of the ultrasound measurement. In alternative embodiments, the ultrasound transducer 200 may be made movable so that it can be pushed to the surface of the elongate sensor carrier 110 during ultrasound image acquisition and retracted at other times, such as when DOT imaging light is being generated and acquired. Thus, when the transrectal diagnostic device 100 is used on a patient, the matching gel may be placed into the transducer window 201 prior to insertion of the device into the patient's rectum. As discussed below, it may also be injected in place after insertion.

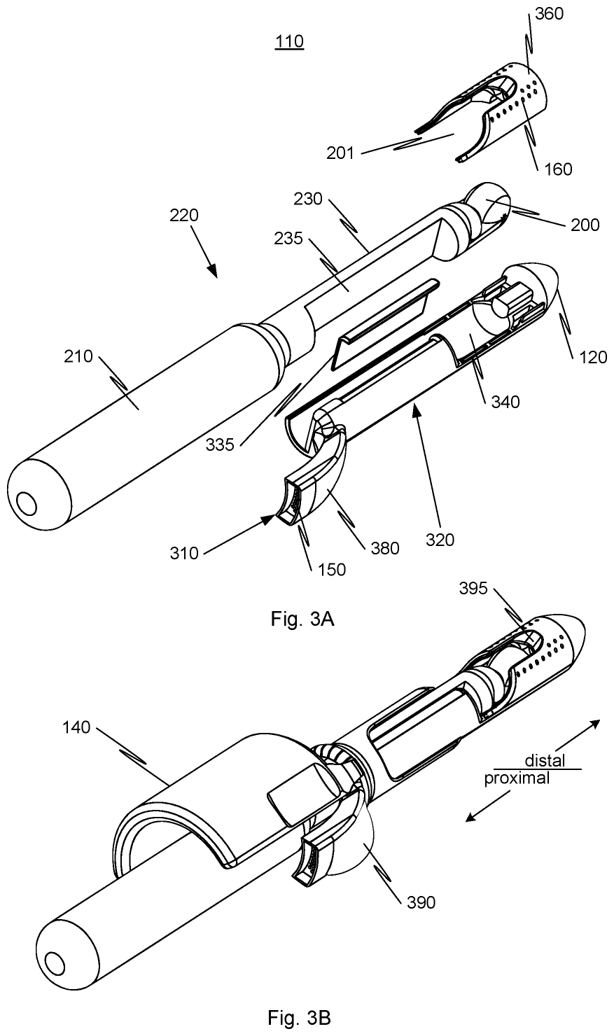

As mentioned, in embodiments, as shown in FIG. 3B, a reservoir such as a bladder 390 filled with matching gel is provided on the handle 380 or at some other location. This may permit the matching gel to be initially applied, or replenished during use, to the transducer window 201. When the bladder 390 is squeezed, gel dispenses through a channel (not shown) embedded in the walls of the optical probe chassis 320, or running through it, to a matching gel channel outlet 395 so that it fills the volume space adjacent the window 201.

While the color of the transducer 200 may not affect its performance for ultrasound scanning, it may be advantageous to use a light-absorbing material, coating, or paint on the surface of the transducer 200. For example, black paint or another dark color may be applied. This may be applied near the fiber distal ends 160 or the entirety of the elongate sensor carrier 110 to minimize optical reflections from the transducer 200 when it is used.

Referring also to FIG. 3A, an exploded view of the elongate sensor carrier 110 is shown. As noted above, the elongate sensor carrier 110 carries both acoustic sensors (ultrasound transducer) and optical sensors (optical fibers that transmit light and receive reflected and refracted light). DOT imaging of the prostate and its surroundings can be performed with a continuous wave optical tomography imaging system. Other types of DOT imaging are known and, as will be evident from the disclosure, may be also be used. The imaging system may employ multiple laser diodes generating multiple different wavelengths. In one example, two laser diodes can be used at wavelengths λ=765 nm and 830 nm as light sources. The system may employ a digital lock-in detection technique as known in the art. Each diode may be modulated to distinct amplitude and frequency as well. Using multiple source fiber bundles, the multi-wavelength sources are sequentially guided to the prostate area through the inner rectal wall.

In an embodiment, 11 fiber bundles are used to convey light for illumination of the test subject, with each bundle being 1 mm in diameter and consisting of 9 multimode plastic fibers in a thin polyimide jacket to limit the bending radius. An example of such fibers is manufactured by Fiberoptic Systems, Inc. As shown in FIG. 3A, the optical fibers 150 terminate at the optical fiber connector 310 in a handle 380. The handle 380 extends radially away from the probe chassis 320, and substantially in a proximal direction (i.e., away from the distal end 120). The handle 380 may extend diagonally from the radial direction toward the proximal direction. The fibers 150 may pass through the probe chassis 320. As such, they are not shown in FIG. 3A, except for their termination in the optical fiber connector 310. The fibers 150 extend toward distal end 120 and curve toward multiple apertures 160 in the optical sensor head 360. The number of fiber bundles may be varied. Other types of light guides may be used in place of plastic fiber bundles. The light guides may be integrated in the housing of the elongate sensor carrier 110. The multiple apertures 160 may be regularly spaced in a regular array or may be distributed in other patterns as known in the art. In embodiments, the apertures 160 (or diffusers) may be spaced apart by a minimum or constant distance, for example, the spacing may be 4 mm on-centers. Note that the term aperture here is understood to be an opening that exposes the tip of a fiber or fiber bundle. Note that the term “fiber” as used herein may connote a single optical fiber or a bundle thereof.

The probe chassis 320 is generally cylindrical in shape, with a transducer cavity 340 formed in the interior. The transducer cavity 340 has a shape and size to accommodate the transducer 200 of the ultrasound wand 220, as will be discussed in greater detail below. The probe chassis 320 has an additional internal cavity covered by cover plate 335 that has a flat face. The flat face of the cover plate 335 creates a flat surface inside of probe chassis 320, such that the ultrasound wand 220, which also has a flat surface 235, can mate with probe chassis 320. When the ultrasound wand 220 and the probe chassis 320 are positioned next to each other, with the flat surface 235 of the ultrasound wand 220 in direct contact with the flat surface of the cover plate 335, the ultrasound wand 220 and the probe chassis 320 can be considered one monolithic bi-modal sensor assembly that provides both ultrasound sensing and optical tomography sensing capability.

As shown in FIG. 3A, the ultrasound wand 220 also includes a handle 210, which may have a larger diameter than a narrow portion 230. The handle 210 may contain electronics and control circuitry for the transducer 200. The narrow portion 230 has the flat surface 235 on one side, as described above. The flat surface 235 does not extend through the entirety of the narrow portion 230, but rather forms a notch that is keyed to the corresponding shape of the probe chassis 320, so that the ultrasound wand 220 and the probe chassis 320 are interferingly engaged with each other to form the elongate sensor carrier 110, which inserted into a probe carrier 140.

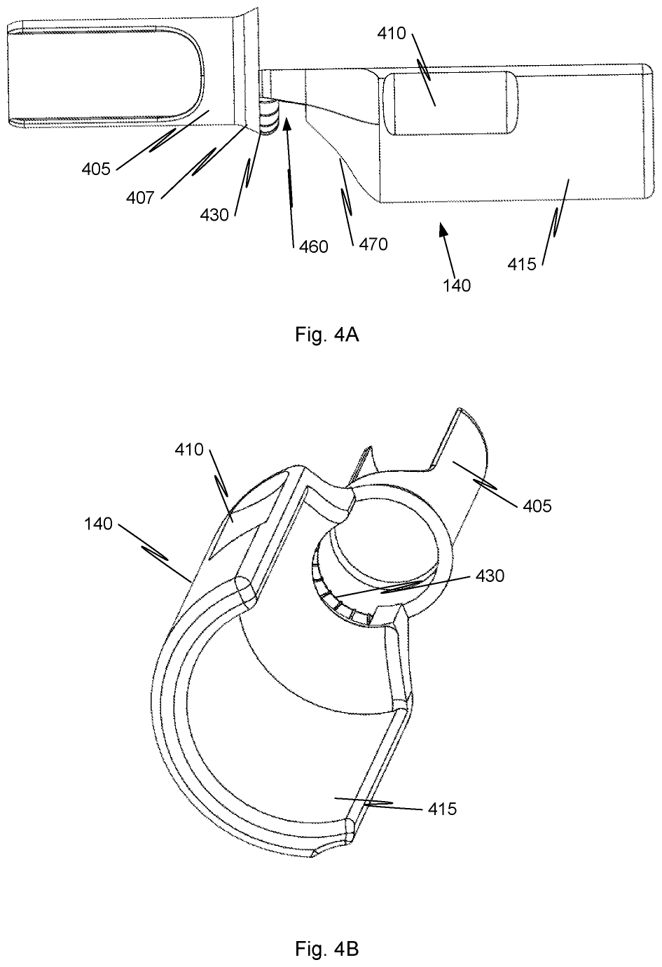

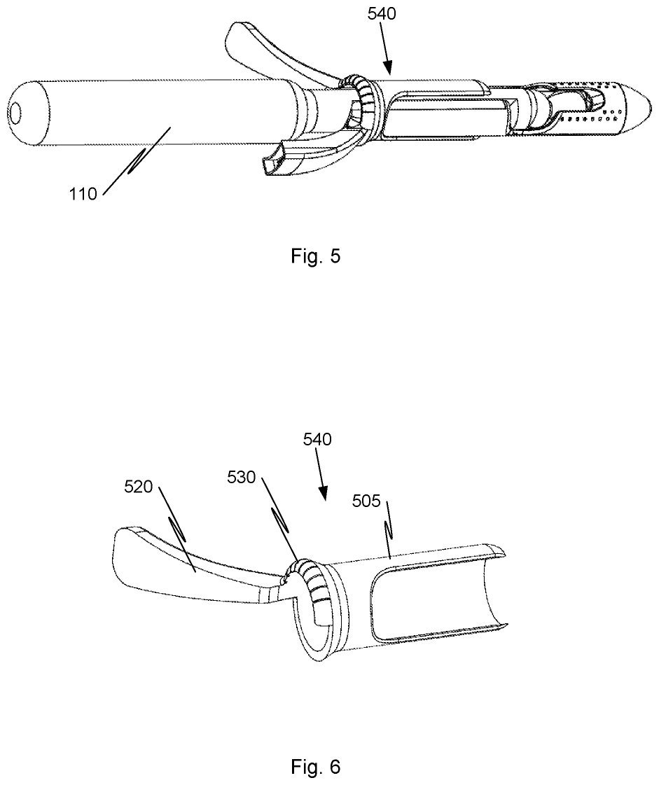

An embodiment of the probe carrier 140 is illustrated in FIGS. 1 through 4B. An alternative embodiment of the probe carrier 140 is shown in FIGS. 5 and 6. Referring to FIG. 4A, the probe carrier 140 has a narrow portion 405 at its distal end and a proximal portion 415 at its proximal end. The inner diameter of the narrow portion 405 has a size that matches the outer diameter of the body of probe chassis 320, such that the elongate sensor carrier 110 can be inserted into the narrow portion 405 of probe carrier 140. In an embodiment, the outer diameter of the body of the probe is 28 mm The shape of the opening in the narrow portion 405 is circular, which permits the rotation of the elongate sensor carrier 110 when it is inserted into the probe carrier 140, as shown in FIG. 1.

The narrow portion 405 continues to an enlarged flange 407 at approximately the midpoint of the probe carrier 140. The enlarged flange 407 terminates with a rim 430 which contains a number of angle markings. In some embodiments, the angle markings may be molded into the probe carrier 140 for example as notches in the rim 430. As shown in FIG. 4B, the notched rim 430 extends partly around the entire enlarged flange 407 so that it extends over a range of motion of the elongate sensor carrier 110 relative to the probe carrier 140. Thus, the angular extent of the rim 430 may be limited to correspond with the range of rotation available for elongate sensor carrier 110 relative to the probe carrier 140. In embodiments, the angular range is between 90 and 180 degrees. In embodiments, the angle markers are 15 degrees apart. In specific embodiments, the angular range is 120 degrees.

While not shown in FIG. 4A, a pointer may be provided in or adjacent to the window 460 to indicate the angular position. In embodiments, an encoder may be connected to the probe carrier 140 and engaged by the elongate sensor carrier 110 to generate an angle signal that may be applied to the controller or a diagnostic computer coupled to the transrectal diagnostic device 100.

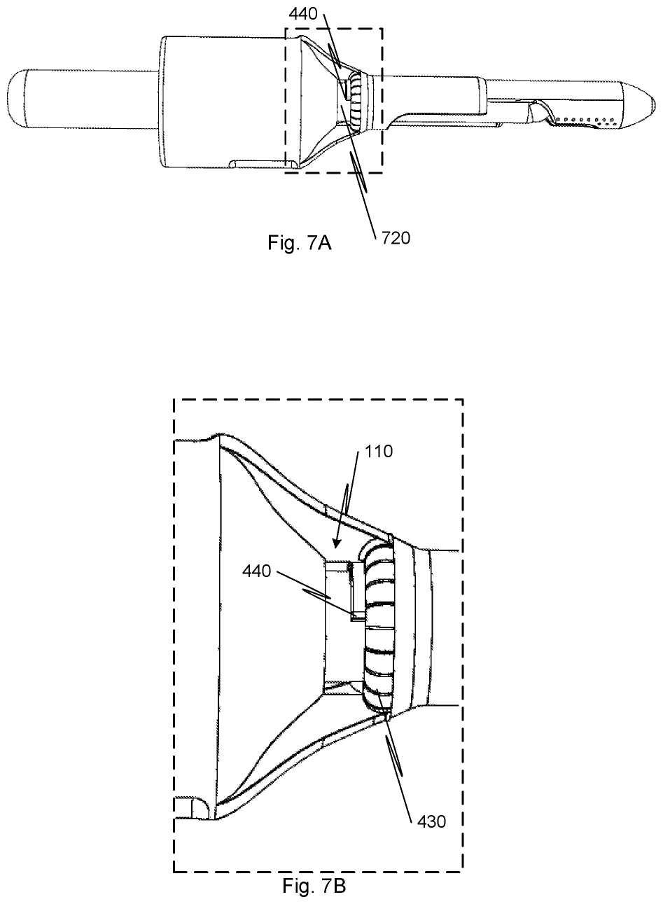

A tapered region 470 extends from the rim 430 to the proximal portion 415 of the probe carrier 140. The tapered region 470 has a curvature and size intended to improve patient comfort when the rotatable probe is inserted into the patient's rectum, with the tapered region 470 coming to rest against the buttocks. A window 460 is disposed between the tapered region 470 and the rim 430, providing a view of the angle markers and an angle indicator 720 on the elongate sensor carrier 110. A view through window 460 is shown in FIG. 7B, described in more detail below.

The proximal portion 415 has a hemi-cylindrical shape, as shown in FIG. 4B, and includes handling recesses 410 to help the clinician grasp the transrectal diagnostic device 100 when the elongate sensor carrier 110 is inserted into the probe carrier 140. The outer dimension of the proximal portion 415 are selected for patient comfort and to provide a secure grip for the clinician. As can be appreciated, the transrectal diagnostic device 100 may have a set of differently sized probe carriers 140 to accommodate different patients. Note that narrow portion 405 is shaped and sized to permit insertion into the rectum.

An alternate embodiment of the probe carrier 540 is illustrated in FIGS. 5 and 6. As shown in FIG. 5, the transrectal diagnostic device 100 still includes the same elongate sensor carrier 110, but the probe carrier 540 differs from the probe carrier 140. An exemplary embodiment of probe carrier 540 has an elongate narrow portion 505 extending from a distal end to a proximal edge 530. The proximal edge 530 also has a set of angle markers disposed along a portion thereof, much like rim 430. A handle 520 extends from edge 530 radially out and curved toward the proximal direction. The functionality of the transrectal diagnostic device 100 is substantially similar when the probe carrier 540 as when carrier 140 is used, and already described above.

Referring to FIG. 7A, the angle indicator 720 includes the angle markers on the rim 430 and pointer 440 formed in the elongate sensor carrier 110. When the optical probe chassis 320 rotates inside of the when carrier 140, the pointer of the optical probe body 440 slides past the angle markers on the rim 430. FIG. 7B shows a close-up view of a portion of FIG. 7A, with the pointer of the elongate sensor carrier 110 shown between two angle markers of rim 430.

In some embodiments, the edge of the optical probe body 440 may have a shape or a structure that physically interacts with the angle markers, providing tactile or audible feedback as a détente mechanism. The physical interaction may also lock the elongate sensor carrier 110 at a particular angular rotation relative to the when carrier 140, reducing movement and jitter that could adversely affect the scanning performance of the optical and/or ultrasound sensor. In other words, the elongate sensor carrier 110 may rotate from one position to the next position and lock in place due to the physical interaction. When sufficient force is applied to overcome the physical interaction, the elongate sensor carrier 110 rotates to the next angular position, where it is again locked into place. This repeats for all angular markers, giving the clinician time to take clean readings at each angle.

Having described the general structure of the transrectal diagnostic device 100, a method of using it follows. The transrectal diagnostic device 100 is assembled such that elongate sensor carrier 110 is inserted into a probe carrier 140, but it is understood that this approach applies equally to the probe carrier 540. An amount of matching gel is placed into the transducer window 201, and the device (enclosed in a hygienic protector) is inserted into the patient's rectum until the optical sensor head 360 is substantially adjacent to the location of the prostate. The handle 380 of the optical probe chassis 320 is rotated to one extreme range of rotation within the probe carrier 140 either before or after insertion into the patient, and a reading with the ultrasound and the optical sensor is collected.

The probe carrier 140 serves as an angular frame of reference, so the probe carrier 140 remains stationary throughout the procedure. After the first reading is completed, the elongate sensor carrier 110 is rotated within the probe carrier 140. Typically, the rotation amount corresponds to one angle marker on the rim 430, and a second ultrasound and optical reading is taken. This process is repeated for the whole angular rotation range noted above (e.g., 120 degrees), or some subset thereof.

Another embodiment of the transrectal diagnostic device is an integrated bimodal probe 800 that includes an elongate sensor carrier 820 that integrates optical tomography transducer and ultrasound transducer elements. As shown in FIG. 8, the integrated bimodal probe 800 has an outer handle 810 that may be held and manipulated the clinician using the integrated bimodal probe 800. The outer handle 810 surrounds the elongate sensor carrier 820, whose housing lies partially within the outer handle 810. The entire integrated bimodal probe 800 is elongate and substantially cylindrical. The elongate sensor carrier 820 has a tapered distal end to ease the insertion of the elongate sensor carrier 820 into the patient's rectum.

Referring still to FIG. 8, the elongate sensor carrier 820 has on it several sources and detectors 830 (or optical apertures). The sources and detectors 830 are coupled to optical fibers that lead to a light source or a light detector. Typically, half of the sources and detectors 830 serve as sources and the other half serve as detectors for performing diffuse optical tomography.

In an embodiment, the sources and detectors 830 surround two ultrasound transducer windows 841, 842. In embodiments, a biopsy needle window 850 may also be provided near the sources and detectors 830 and the transducer windows 841, 842. The transducer windows 841, 842 may be positioned to provide room for the transducer windows 841, 842. Ultrasound transducers 941 and 942 may be carried on the end of a movable ultrasound transducer assembly 943 which may be movable or tiltable to cause the Ultrasound transducers 941 and 942 to move toward the surface of the elongate sensor carrier 820 such that its transducers are aligned with the surface, or protrude from the surface of the elongate sensor carrier 820.

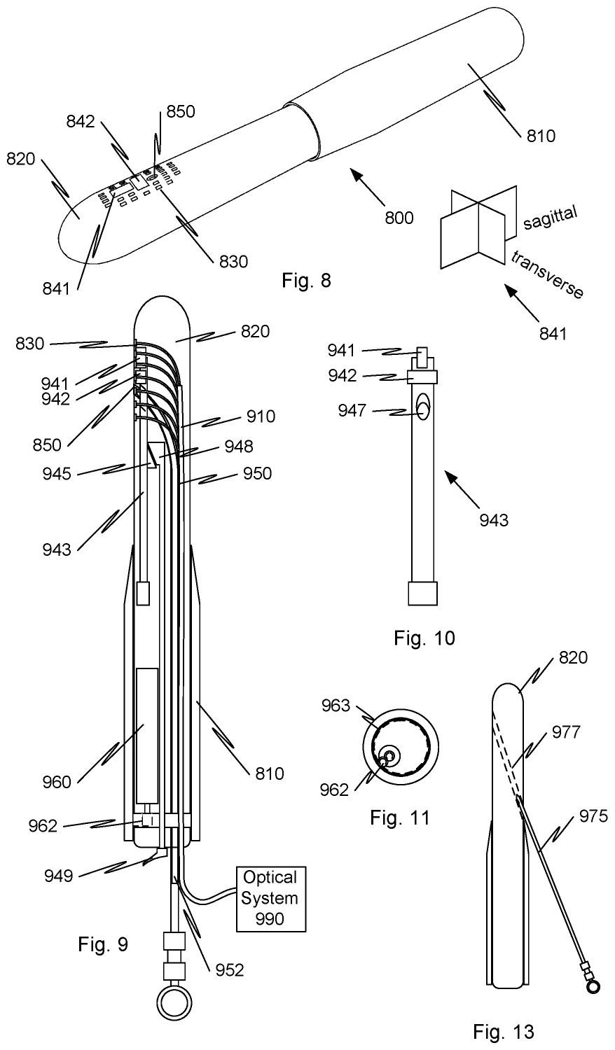

FIG. 10 illustrates an embodiment of the ultrasound transducer assembly 943 that is otherwise shown inside the integrated bimodal probe 800. The ultrasound transducer assembly 943 has an elongate, rod-shaped body with sagittal ultrasound transducer 941 at the distal end of the ultrasound transducer assembly 943, and a transverse ultrasound transducer 942 next to the sagittal ultrasound transducer 941, but closer to the proximal end of the ultrasound transducer assembly 943. As seen in FIG. 10, the sagittal ultrasound transducer 941 and the transverse ultrasound transducer 942 have generally rectangular profiles, but orthogonal orientation relative to each other to permit sagittal and transverse ultrasound sections (sagittal and transverse planes being referenced to the body of the integrated bimodal probe 800 as indicated at 841). While not visible in FIG. 10, the ultrasound transducer assembly 943 may also include a ramp 945, as shown in FIG. 9.

Referring to FIG. 9, the transducers 941 and 942 are shown in a position retracted into the elongate sensor carrier 820, though accessible through windows 841 and 842. As known, it is advantageous for ultrasound transducers to be in contact with the tissue being imaged or to provide an acoustically transparent medium between the transducers and the inspected tissue. Thus, the transducers 941 and 942 may be movable in the radial direction of the elongate sensor carrier 820 such that they can extend and retract relative to the windows 841 and 842. One approach for presenting the transducers 941 and 942 through the windows is the ramp 945 on the ultrasound transducer assembly 943 and a transducer positioning controller 948. The transducer positioning controller 948 can be thought of as a transfer bar whose movement is controlled by a control handle 949. When the handle 949 is pulled in the proximal direction, it slides an incline against the ramp 945, thereby pressing the whole ultrasound transducer assembly 943 radially outward, causing the transducers 941 and 942 to extend to the surface or beyond the surface of the elongate sensor carrier 820. Alternatively, a matching gel may be injected adjacent the windows as discussed above in connection with the various embodiments.

It will be understood that the handle 949 can be eliminate and replaced with a mechanical linkage that is controlled by the diagnostic system to reduce the manual work required of the clinician. The mechanical linkage may be powered by actuator (or motor) 960, or by a separate, dedicated actuator. In an embodiment, the actuator 960 controls the rotation of the elongate sensor carrier 820 relative to the outer handle 810. As the outer handle 810 is held in place, the actuator 960 may turn a gear 962, which rotates along a geared ring 963 with internal teeth, as shown in FIGS. 9 and 11. The actuator 960 may output a signal representative of the rotation angle, or a separate angle measurement may be conducted by an encoder, similarly to the embodiments discussed above.

The actuator 960 can be a stepper motor, or another type of an electrical motor. While the actuator 960 is illustrated as being attached to the outer handle 810 in FIG. 9, it could also be attached to the sensor carrier 820 and still drive the rotation of the sensor carrier 820 relative to the outer handle 810. While the embodiment in FIG. 3B does not illustrate a motor or actuator, one could be provide either on the sensor carrier 110 or on the probe carrier 140 to effect the rotation of the sensor carrier 110 relative to the probe carrier 140 like in the embodiment of FIG. 9. The motor could also drive to rotation of the sensor carrier 110 through a continuous range, rather than discrete steps, to effect a continuous scan of the tissue, rather than discrete slices. It will be understood that the actuator 960 in FIG. 9 could also continuously rotate the sensor carrier 820 to perform such a scan.

While not illustrated in the figures, the actuator 960 may be replaced by a mechanical linkage such as a flex-drive that has a flexible rotating shaft inside a flexible conduit, such that the rotational force is provided from outside of the probe. In this case, the overall size and weight of the probe can be reduced. The flex-drive can have a connectable link at the proximal end of the probe so that the flexible conduit can be easily connected and disconnected.

Referring to FIG. 9, cross-sectional view along the rotational axis of the integrated bimodal probe 800 is shown. A fiber bundle 910 runs from the sources and detectors 830, through the elongate sensor carrier 820 to optical system 990. Although not shown in the drawing, a connector or a plug can be located at the proximal end of the integrated bimodal probe 800 such that the probe can be easily disconnected from the optical system 990.

In an embodiment, the integrated bimodal probe 800 may be used to collect a biopsy sample with a flexible biopsy needle 952 integrated into the probe. As shown in FIG. 9, the flexible biopsy needle 952 extends from the proximal end of the integrated bimodal probe 800 toward the distal end. In some embodiments the flexible biopsy needle 952 might not reach back to the proximal end, and may instead be embedded in a flexible extension material that allows the needle to be pressed through the biopsy needle sleeve 950. When the flexible biopsy needle 952 is present, the ultrasound transducer assembly 943 may have a hole 947 therein to accommodate the needle passing through the ultrasound transducer assembly 943. The elongate sensor carrier 820 may also have a biopsy needle window 850 that provides an opening for the needle to be extended into the tissue of interest (once the tissue has been identified and observed with ultrasound and DOT). It would be understood that the biopsy needle need not be present in all embodiments, and that the various needle holes can then be omitted.

FIG. 13 shows an alternative device for permitting the use of a biopsy needle in connection with any of the probe embodiments disclosed. A probe 820 transducer carriers has a diagonal sleeve indicated at 820 that opens on opposite sides of the probe transducer carrier. A separate biopsy needle 975 may be inserted through the diagonal sleeve 977 so that it emerges into the tissue being imaged. The biopsy needle 975 may be inserted after the probe 820 is positioned in the patient.

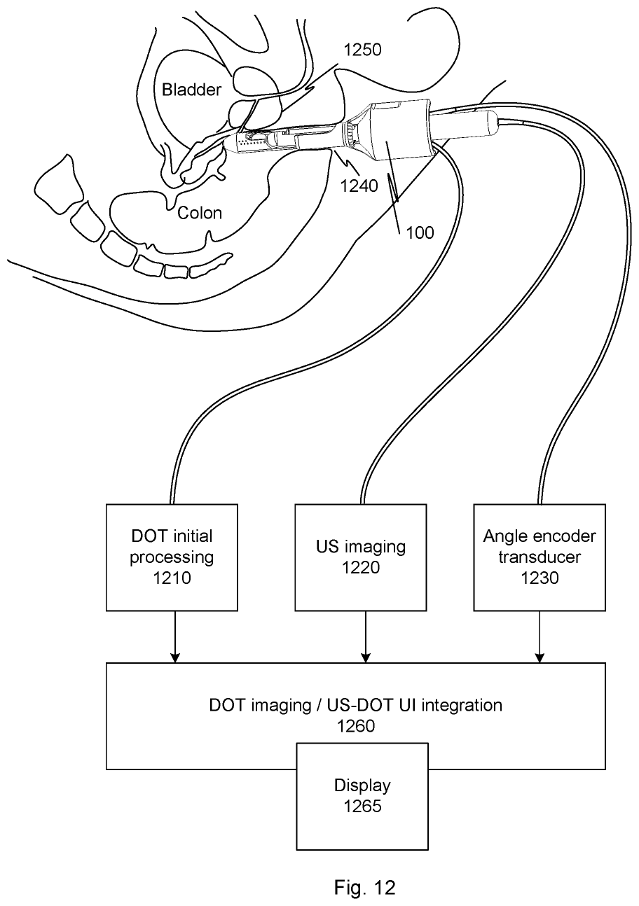

Referring to FIG. 12, an exemplary embodiment of a combined imaging system may use a transrectal diagnostic device as described in the various embodiments above to provide diagnostic information to a clinician. While this discussion refers to the transrectal diagnostic device 100, it will be understood that the integrated bimodal probe 800 may be used. The transrectal diagnostic device 100 is inserted into the rectum and may be operatively connected to a DOT control and interface processor 1210 via optical fiber or other suitable connection. The DOT control and interface processor 1210 controls the DOT imaging of the transrectal diagnostic device 100. The DOT control and interface processor 1210 may also control the ultrasound imaging functionality, or the ultrasound may also be controlled, at least in part, by the ultrasound imaging processor 1220, which may be operatively connected to the transrectal diagnostic device 100 via a suitable connection such as a wire, a cable, or a wireless connection.

This connection between the ultrasound imaging processor 1220 and the transrectal diagnostic device 100 may carry raw data from the ultrasound transducers, or processed data, as would be the case if some of the processing takes place in the ultrasound wand itself, as noted above. The ultrasound imaging processor 1220 may receive raw data and process it into image data that can be interpreted by a clinician. The ultrasound imaging processor 1220 may also output signals and other data that is received and further processed by the ultrasound-DOT integration processor 1260. For example, the ultrasound imaging processor 1220 may output data that the ultrasound-DOT integration processor 1260 can interpret as surface shape or boundaries of tissue being imaged by the DOT to provide faster and/or more accurate analysis of the DOT data and produce information that can be interpreted by the clinician.

The transrectal diagnostic device 100 may also include an angle encoder, as described above, that outputs a signal representative of the angle of rotation of the sensor carrier 110. The signal representative of the angle may be output via a wired or wireless connection to an angle encoder transducer 1230.

As shown in FIG. 12, the ultrasound-DOT integration processor 1260 may receive data from the DOT control and interface processor 1210, the ultrasound imaging processor 1220, and the angle encoder transducer 1230. In various embodiments, only a subset of the connections may be used. The connections between the various processors may be bi-directional, as shown in FIG. 12, such that control information can pass from the ultrasound-DOT integration processor 1260 to the other processors. For example, angle information from the angle encoder transducer 1230 may pass through the ultrasound-DOT integration processor 1260 to the DOT control and interface processor 1210. The DOT control and interface processor 1210 may use the angle information to process raw data or combine it with processed data and save it for further processing and rendering for the clinician. In other embodiments, the encoder transducer 1230 may output a driving signal to the transrectal diagnostic device 100 which effects the rotation of the sensor carrier based on the driving signal. In this way, the entire scanning process can be automated, or semi-automated, after the transrectal diagnostic device 100 is inserted into the patient, with automated rotation and optical and ultrasound scanning taking place under the control of the ultrasound-DOT integration processor 1260 and/or one or more of the other processors 1210, 1220, and 1230.

The ultrasound-DOT integration processor 1260 may process the data it receives from the different processors 1210, 1220, and/or 1230 to render a graphical representation of the tissue imaged by the transrectal diagnostic device 100 and output the graphical representation to a display 1265. A clinician interprets the information to determine or refine a clinical diagnosis.

According to embodiments of the disclosure, a transrectal diagnostic device includes an elongate sensor carrier having at least one ultrasound transducer located adjacent an ultrasound transducer window proximate a distal end of the elongate sensor carrier. Optical fibers terminate at distal ends thereof at multiple points around the ultrasound transducer window and the optical fibers optical connector terminates at proximal ends thereof at an optical connector. Further, the transrectal diagnostic device includes a probe carrier to which said elongate sensor carrier is rotatably connected, the probe carrier and sensor carrier each having a respective part of an indicator that indicate a rotational position of the sensor carrier relative to the probe carrier.

In some embodiments, the indicator may include an encoder and the respective parts include relatively movable parts of an optical encoder. The indicator may also include an encoder and the respective parts may include a first element with an array of regularly spaced angle marks and a second element carrying a pointer positioned to point to the angle marks.

In some embodiments, the probe carrier may have a first handle that extends radially from a longitudinal axis of the elongate sensor carrier. The first handle may be curved such that at a base thereof, its radially-outer surface is parallel to a said longitudinal axis and at a point remote from the base, the radially outer surface is within 45 degrees of a line perpendicular to the longitudinal axis.

In some embodiments, the optical connector may be positioned at the end of a second handle stemming from the elongate sensor carrier, and the transrectal device may also include an ultrasound processor connected to the at least one ultrasound transducer.

According to further embodiments, the transrectal diagnostic device may include an optical tomography control and interface processor that contains light sources and light detectors connected to the optical connector that controls light detected and output by the optical fibers. The optical tomography control and interface processor converts output from the optical detectors to data and provides the data to an optical tomography imaging processor. The imaging processor receives ultrasound image data which is combined with optical tomography data to form optical tomography images, the ultrasound image data providing anatomical boundary conditions for the optical tomography solutions computed by the optical tomography processor.

According to further embodiments, the probe carrier has a cylindrical portion that extends partly along the elongate sensor carrier. The at least one ultrasound transducer includes a first and second ultrasound transducer, the first being oriented for making a slice in the sagittal plane and the second being oriented for making a slice in the transverse plane where sagittal and transvers are taken with reference to the elongate sensor. The probe carrier may have a hollow hemi-cylindrical body.

According to further embodiments of the transrectal diagnostic device, one of the elongate sensor carrier and the probe carrier has fiducial marks to indicate angular position and the other has a pointer positioned to indicate a selected one of the fiducial marks. In some embodiments, one of the elongate sensor carrier and the probe carrier has a motor with a drive that is connected to rotate the elongate sensor carrier and the probe carrier relative to each other. In further embodiments, one of the elongate sensor carrier and the probe carrier has a motor with a drive that is connected to rotate the elongate sensor carrier and the probe carrier relative to each other in a progressive motion to perform a scan.

According to further embodiments, the transrectal diagnostic device is part of a system that includes an optical tomography control and interface processor that convert output from the optical detectors to data and provide the data to an optical tomography imaging processor. Further, the ultrasound imaging processor receives ultrasound image data which is combined with optical tomography data to form optical tomography images, the ultrasound image data providing anatomical boundary conditions for the optical tomography solutions computed by the optical tomography processor. The optical tomography control and interface processor controls the ultrasound imaging processor. In some embodiments, the optical tomography control and interface processor acquire ultrasound and optical tomography illumination data simultaneously.

According to further embodiments, an optical probe system includes a body configured to accommodate a plurality of source and detector fiber bundles, the body including a cavity configured to accommodate an ultrasound probe. The system may further include a holder configured to receive the body and allow the body to rotate about an axis when inserted into the holder. In some embodiments, the system also includes the ultrasound probe inserted into the cavity of the body. The ultrasound probe may be a TRUS probe.

In some embodiments, the body has cylindrical shape extending along a central axis, and the cavity extends along the central axis from one end of the body and opens in a radial direction. The body may include at least 11 source fiber bundles and at least 11 detector fiber bundles. In some embodiments, the body includes more than 11 source fiber bundles and more than 11 detector fiber bundles.

According to further embodiments, the optical probe system may include a rotational actuator configured to rotate the body within the holder. In some embodiments, the body includes a window configured to accommodate a transducer of the ultrasound probe. The body may further include a hole for a biopsy needle.

In some embodiments, the optical probe system includes an array of active pixel sensors disposed at an end of the body. The system may also include a light source positioned adjacent to the array.

According to further embodiments, a transrectal diagnostic device includes an elongate sensor carrier having at least one ultrasound transducer located adjacent an ultrasound transducer window proximate a distal end of the elongate sensor carrier and light sources and detectors terminating at distal ends thereof at multiple points around the ultrasound transducer window. The device also includes a probe carrier to which said elongate sensor carrier is rotatably connected, the probe carrier and sensor carrier each having a respective part of an indicator that indicate a rotational position of the sensor carrier relative to the probe carrier.

In some embodiments, the least one of the probe carrier and the sensor carrier has a handle. At least one of the probe carrier and the sensor carrier may have a curved handle. In some embodiments, at least one of the probe carrier and the sensor carrier has a handle carrying a connector. The indicator may include an encoder and the respective parts include relatively movable parts of an optical encoder. In some embodiments, the respective parts include relatively movable parts of a resistance encoder. In some embodiments, the respective parts include a first element with an array of regularly spaced angle marks and a second element carrying a pointer positioned to point to the angle marks. The probe carrier may have a first handle that extends radially from a longitudinal axis of the elongate sensor carrier. The first handle may be curved such that at a base thereof, its radially-outer surface is parallel to a said longitudinal axis and at a point remote from the base, the radially outer surface is within 45 degrees of a line perpendicular to the longitudinal axis. In some embodiments, the optical connector is positioned at the end of a second handle stemming from the elongate sensor carrier. An ultrasound processor may be connected to the at least one ultrasound transducer. The transrectal diagnostic device may include an optical tomography control and interface processor that contains light sources and light detectors connected to the optical connector that controls light detected and output by the optical fibers. In some embodiments the optical tomography control and interface processor converts output from the optical detectors to data and provides the data to an optical tomography imaging processor. The imaging processor may receive ultrasound image data which is combined with optical tomography data to form optical tomography images, the ultrasound image data providing anatomical boundary conditions for the optical tomography solutions computed by the optical tomography processor.

In some embodiments, the probe carrier has a cylindrical portion that extends partly along the elongate sensor carrier. In some embodiments, the at least one ultrasound transducer includes a first and second ultrasound transducer, the first being oriented for making a slice in the sagittal plane and the second being oriented for making a slice in the transverse plane where sagittal and transvers are taken with reference to the elongate sensor. Further, the probe carrier may have a hollow hemi-cylindrical body.

According to further embodiments, one of the elongate sensor carrier and the probe carrier has fiducial marks to indicate angular position and the other has a pointer positioned to indicate a selected one of the fiducial marks. In some embodiments, one of the elongate sensor carrier and the probe carrier has a motor with a drive that is connected to rotate the elongate sensor carrier and the probe carrier relative to each other. In other embodiments, one of the elongate sensor carrier and the probe carrier has a motor with a drive that is connected to rotate the elongate sensor carrier and the probe carrier relative to each other in a progressive motion to perform a scan. Further, the optical tomography control and interface processor may convert output from the optical detectors to data and provides the data to an optical tomography imaging processor, and the ultrasound imaging processor may receive ultrasound image data which is combined with optical tomography data to form optical tomography images, the ultrasound image data providing anatomical boundary conditions for the optical tomography solutions computed by the optical tomography processor. Further, the optical tomography control and interface processor may control the ultrasound imaging processor. Furthermore, the optical tomography control and interface processor acquire ultrasound and optical tomography illumination data simultaneously.

According to further embodiments, the disclosed subject matter includes a transrectal diagnostic device. An elongate sensor carrier has an array of sources and detectors for an optical tomography imager aimed radially on a surface thereof toward a distal end thereof. The array sources and detectors are spaced apart at a distal end of the sensor carrier. The sensor carrier has a rounded blunt tip to facilitated insertion in the rectum of a patient. At least one ultrasound transducer is positioned at said distal end. In variations, a probe carrier is rotatably coupled to the elongate sensor carrier. In further variations, a probe carrier is rotatably coupled to the elongate sensor carrier and a motor drive to generate a torque to rotate the probe carrier relative to the elongate sensor carrier according to commands from a controller. In further variations, a probe carrier may be rotatably coupled to the elongate sensor carrier and a motor drive to generate a torque to rotate the probe carrier relative to the elongate sensor carrier according to commands from a controller, wherein the controller generates a progressive sweep of the ultrasound transducer and sources and detectors to interrogate a predefined volume and samples data during the sweep. At least one of the probe carrier and the sensor carrier may have a curved handle. At least one of the probe carrier and the sensor carrier may have a handle carrying a connector. The probe carrier and sensor carrier may each have a respective part of an indicator that indicate a rotational position of the sensor carrier relative to the probe carrier, the indicator including an encoder and the respective parts include relatively movable parts of an optical encoder. The probe carrier and sensor carrier may each have a respective part of an indicator that indicate a rotational position of the sensor carrier relative to the probe carrier, the indicator including an encoder and the respective parts include relatively movable parts of a resistance encoder. The probe carrier and sensor carrier may each have a respective part of an indicator that indicate a rotational position of the sensor carrier relative to the probe carrier, the indicator including an encoder and the respective parts include a first element with an array of regularly spaced angle marks and a second element carrying a pointer positioned to point to the angle marks. The probe carrier may have a first handle that extends radially from a longitudinal axis of the elongate sensor carrier. The first handle is curved such that at a base thereof, its radially-outer surface is parallel to a said longitudinal axis and at a point remote from the base, the radially outer surface is within 45 degrees of a line perpendicular to the longitudinal axis. The optical connector may be positioned at the end of a second handle stemming from the elongate sensor carrier. An ultrasound processor may be connected to the at least one ultrasound transducer. An optical tomography control and interface processor that contains light sources and light detectors may be connected to the optical connector that controls light detected and output by the optical fibers. The optical tomography control and interface processor may convert output from the optical detectors to data and provides the data to an optical tomography imaging processor. The imaging processor may receive ultrasound image data which are combined with optical tomography data to form optical tomography images, the ultrasound image data providing anatomical boundary conditions for the optical tomography solutions computed by the optical tomography processor. The probe carrier may be shaped to have a cylindrical portion that extends partly along the elongate sensor carrier. The at least one ultrasound transducer may include a first and second ultrasound transducer, the first being oriented for making a slice in the sagittal plane and the second being oriented for making a slice in the transverse plane where sagittal and transvers are taken with reference to the elongate sensor. The probe carrier may have a hollow hemi-cylindrical body. One of the elongate sensor carrier and the probe carrier may have fiducial marks to indicate angular position and the other has a pointer positioned to indicate a selected one of the fiducial marks. One of the elongate sensor carrier and the probe carrier may have a motor with a drive that is connected to rotate the elongate sensor carrier and the probe carrier relative to each other. One of the elongate sensor carrier and the probe carrier may have a motor with a drive that is connected to rotate the elongate sensor carrier and the probe carrier relative to each other in a progressive motion to perform a scan.

Any of the foregoing embodiments may be modified to add the permissive limitations in any combination to form additional embodiments. These combinations are not disclosed explicitly because it would be render the specification itinerant and diffuse. However, such variations are readily apparent from the disclosure and claims clearly fall within the scope of the enabling disclosure as well as the original written description herein. For example such embodiments include every conceivable combination, except as contradictory, of the dependent claims not explicitly combined in a claim.

Features of the disclosed embodiments may be combined, rearranged, omitted, etc., within the scope of the disclosed subject matter to produce additional embodiments. Furthermore, certain features may sometimes be used to advantage without a corresponding use of other features. It is, thus, apparent that there is provided, in accordance with the present disclosure, a needle guard and associated manufactures, components, systems, and methods of use. Many alternatives, modifications, and variations are enabled by the present disclosure. While specific embodiments have been shown and described in detail to illustrate the application of the principles of the disclosure, it will be understood that the disclosed subject matter may be embodied otherwise without departing from such principles. Accordingly, Applicants intend to embrace all such alternatives, modifications, equivalents, and variations that are within the spirit and scope of the present disclosure.

1. A transrectal diagnostic device, comprising:
an elongate sensor carrier having at least one ultrasound transducer located adjacent an ultrasound transducer window proximate a distal end of the elongate sensor carrier;
optical fibers terminating at distal ends thereof at multiple points around the ultrasound transducer window;
the optical fibers terminating at proximal ends thereof at an optical connector;
a probe carrier to which said elongate sensor carrier is rotatably connected;
the probe carrier and sensor carrier each having a respective part of an indicator that indicate a rotational position of the sensor carrier relative to the probe carrier.

2. The device of claim 1, wherein the indicator includes an encoder and the respective parts include relatively movable parts of an optical encoder or of a resistance encoder.

3. (canceled)

4. The device of claim 1, wherein the indicator includes an encoder and the respective parts include a first element with an array of regularly spaced angle marks and a second element carrying a pointer positioned to point to the angle marks.

5. The device of claim 1, wherein the probe carrier has a first handle that extends radially from a longitudinal axis of the elongate sensor carrier.

6. The device of claim 5, wherein the first handle is curved such that at a base thereof, its radially-outer surface is parallel to a said longitudinal axis and at a point remote from the base, the radially outer surface is within 45 degrees of a line perpendicular to the longitudinal axis.

7. The device of claim 1, wherein the optical connector is positioned at the end of a second handle stemming from the elongate sensor carrier.

8. (canceled)

9. The device of claim 1, further comprising an optical tomography control and interface processor that contains light sources and light detectors connected to the optical connector that controls light detected and output by the optical fibers.

10. The device of claim 9, wherein the optical tomography control and interface processor converts output from the optical detectors to data and provides the data to an optical tomography imaging processor.

11. The device of claim 10, wherein the imaging processor receives ultrasound image data which is combined with optical tomography data to form optical tomography images, the ultrasound image data providing anatomical boundary conditions for optical tomography solutions computed by the optical tomography processor.

12. (canceled)

13. The device of claim 1, wherein the at least one ultrasound transducer includes a first and second ultrasound transducer, the first being oriented for making a slice in the sagittal plane and the second being oriented for making a slice in the transverse plane where sagittal and transvers are taken with reference to the elongate sensor.

14. (canceled)

15. The device of claim 1 wherein one of the elongate sensor carrier and the probe carrier has fiducial marks to indicate angular position and the other has a pointer positioned to indicate a selected one of the fiducial marks.

16. The device of claim 1, wherein one of the elongate sensor carrier and the probe carrier has a motor with a drive that is connected to rotate the elongate sensor carrier and the probe carrier relative to each other.

17. (canceled)

18. The device of claim 9, wherein:
the optical tomography control and interface processor converts output from the optical detectors to data and provides the data to an optical tomography imaging processor;
an ultrasound imaging processor receives ultrasound image data which is combined with optical tomography data to form optical tomography images, the ultrasound image data providing anatomical boundary conditions for optical tomography solutions computed by the optical tomography processor;
the optical tomography control and interface processor controls the ultrasound imaging processor; and
the optical tomography control and interface processor acquire ultrasound and optical tomography illumination data simultaneously.

19-30. (canceled)

31. A transrectal diagnostic device, comprising:
an elongate sensor carrier having at least one ultrasound transducer located adjacent an ultrasound transducer window proximate a distal end of the elongate sensor carrier;
light sources and detectors terminating at distal ends thereof at multiple points around the ultrasound transducer window;
a probe carrier to which said elongate sensor carrier is rotatably connected;
the probe carrier and sensor carrier each having a respective part of an indicator that indicate a rotational position of the sensor carrier relative to the probe carrier.

32-33. (canceled)

34. The device of claim 31, wherein at least one of the probe carrier and the sensor carrier has a handle carrying a connector.

35. The device of claim 31, wherein the indicator includes an encoder and the respective parts include relatively movable parts of an optical encoder or of a resistance encoder.

36-41. (canceled)

42. The device of claim 31, further comprising:
an optical tomography control and interface processor that contains light sources and light detectors connected to an optical connector that controls light detected and output by optical fibers, wherein
the optical tomography control and interface processor converts output from the optical detectors to data and provides the data to an optical tomography imaging processor, and
the imaging processor receives ultrasound image data which is combined with optical tomography data to form optical tomography images, the ultrasound image data providing anatomical boundary conditions for optical tomography solutions computed by the optical tomography processor.

43-45. (canceled)

46. The device of claim 31, wherein the at least one ultrasound transducer includes a first and second ultrasound transducer, the first being oriented for making a slice in the sagittal plane and the second being oriented for making a slice in the transverse plane where sagittal and transvers are taken with reference to the elongate sensor.

47. The device of claim 31, wherein the probe carrier has a hollow hemi-cylindrical body.

48-76. (canceled)
